(19) United States
(12) Patent Application Publication
(10) Pub. No.: US 2026/0324971 A1
(43) Pub. Date: Oct. 22, 2026

(54) SLIT LAMP MICROSCOPE SYSTEM, COMPUTER-READABLE RECORDING MEDIUM STORING PROGRAM, AND METHOD FOR OPERATING SLIT LAMP MICROSCOPE SYSTEM

(71) Applicant: TOHOKU UNIVERSITY, Sendai-shi (JP)

(72) Inventors: Neelam KAUSHIK, Sendai-shi (JP); Parmanand SHARMA, Sendai-shi (JP); Toru NAKAZAWA, Sendai-shi (JP); Noriko HIMORI, Sendai-shi (JP)

(73) Assignee: TOHOKU UNIVERSITY, Sendai-shi (JP)

(21) Appl. No.: 19/529,505

(22) Filed: Feb. 4, 2026

Related U.S. Application Data
(63) Continuation of application No. PCT/JP2023/028635, filed on Aug. 4, 2023.

(51) Int. Cl. A61B 3/14; A61B 3/135; G06T 7/00

(52) U.S. Cl. CPC A61B 3/14; A61B 3/135; G06T 7/0012; G06T 2207/10056; G06T 2207/20216; G06T 2207/30041

(57) ABSTRACT

A slit lamp microscope system includes: a photographing device that scans an eye with slit light; a memory; and processor circuitry coupled to the memory and configured to analyze an image obtained by the photographing device using Artificial Intelligence (AI), and determine a condition of the eye based on a result of the analysis, wherein the photographing device captures a plurality of images at each of predetermined angles during the scanning at least one way from one end to other end of the eye.

### CROSS-REFERENCE TO RELATED APPLICATION

This application is a continuation application of International Application PCT/JP2023/028635 filed on Aug. 4, 2023 and designated the U.S., the entire contents of which are incorporated herein by reference.

### FIELD

The technique described in this specification relates to a slit lamp microscope system, a computer-readable recording medium having stored therein a program, and a method for operating a slit lamp microscope system.

### BACKGROUND

One of the related methods of checking an eye irradiates an anterior segment of an eye with a slit lamp (slit light) and observes the anterior segment by scanning. An ophthalmologist observes and photographs the cross section of an eyeball using a table-top binocular slit-lamp microscope to diagnose the condition of the eye. A related slit lamp microscope has been large in size and expensive because it requires a precise and complicated optical system. In recent years, there has been proposed a photographing device that is attached to a mobile terminal and can easily photograph an anterior segment of an eye.

For example, Patent Document 1 discloses a close-range photographing device that is detachably mounted on a mobile device provided with a light source and a camera lens for photographing and that observes and photographs the anterior segment and the fundus of an eye by switching a light path from the light source. The close-range photographing device further includes a member that forms a slit light and a slit forming member that forms light that the light source of the mobile terminal emits into slit light. With this configuration, light reflected from the anterior segment of an eye enters the camera lens for photographing provided on the mobile terminal and the reflected light is photographed and stored by the internal application of the mobile terminal.

Patent Document 1: Japanese Laid-open Patent Publication No.2020-038379

Although simple and convenient, the photographing condition of the close-range photographing device of PTL 1 varies with the photographer and the type of mobile terminal to be attached, which makes it difficult to obtain uniform images. Furthermore, as with checking with a related slit lamp microscope, determination of a condition of the eye from a photographed image requires consultation with a specialized physician. In order to detect eye diseases early, it is desirable to utilize a lightweight Artificial Intelligence (AI) algorithm that is capable of recognizing the structure of the eye and accurately determining the condition of the eye with a small number of processes using AI. However, such a device has a problem in that it is very difficult to obtain photographed images that AI can analyze and determine, and this frequently causes errors in detection.

### SUMMARY

According to an aspect of the embodiment, a slit lamp microscope system includes: a photographing device that scans an eye with slit light; a memory; and a processor circuitry coupled to the memory and configured to analyze an image obtained by the photographing device using Artificial Intelligence (AI), and determine a condition of the eye based on a result of the analysis; wherein the photographing device captures a plurality of images at each of predetermined angles during the scanning at least one way from one end to other end of the eye.

### BRIEF DESCRIPTION OF DRAWINGS

FIG. 1 is a block diagram illustrating a slit lamp microscope system according to an embodiment.

FIG. 2A is a schematic diagram illustrating an image capturing slit light applied to an eyeball, and FIG. 2B is a diagram illustrating the eye structure.

FIG. 3 is a diagram illustrating a process in which an analyzing unit extracts a feature from an image obtained by taking an average value of multiple images, using LWBNA_UNet.

FIG. 4 is a diagram illustrating a process in which a determining unit extracts various parameters, FIG. 4A being an average value image, FIG. 4B being a graph plotting a boundary between a cornea and an iris, FIG. 4C being an image representing a result of determining an eye condition.

FIG. 5 is a block diagram illustrating a hardware configuration of a processing device according to the embodiment.

FIG. 6 is a flowchart illustrating an example of a procedure for operating the slit lamp microscope system of the present embodiment.

### DESCRIPTION OF EMBODIMENTS

Hereinafter, a slit lamp microscope system, a program, a non-transitory computer-readable recording medium having stored therein a program, and a method for operating a slit lamp microscope system according to an embodiment will now be described with reference to the accompanying drawings. However, the following embodiment is merely illustrative and is not intended to exclude the application of various modifications and techniques not explicitly described in the embodiment. Each configuration of the embodiment may be variously modified and implemented without departing from the scope thereof and can be selected, omitted, and appropriately combined.

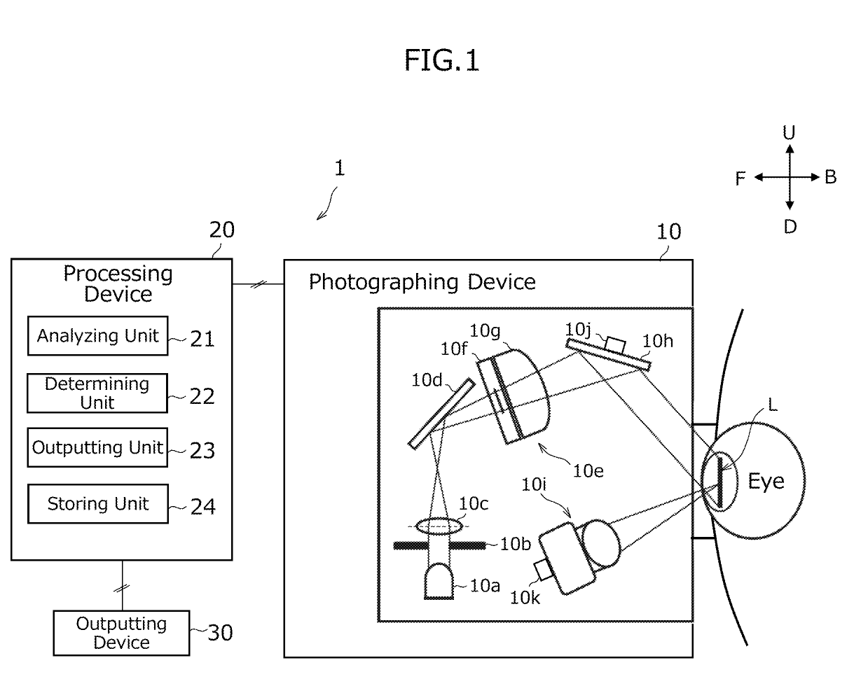

In the present embodiment, the direction in FIG. 1 used for the description is defined as follows. The horizontal direction is a front-rear direction (the front is indicated by “F” and the rear is indicated by “B” in the drawing) and the direction in which gravity acts in the vertical direction is a downward direction (indicated by “D” in the drawing), and the opposite direction to the downward is an upward direction (indicated by “U” in the drawing).

### 1. Configuration

### 1-1. Slit Lamp Microscope System

FIG. 1 is a diagram illustrating a configuration of a slit lamp microscope system 1 according to an embodiment. The slit lamp microscope system 1 according to the present embodiment includes a photographing device 10 and a processing device 20. The photographing device 10 scans an eye with slit light, and captures multiple images at each of predetermined angles during the scanning. The processing device 20 processes images obtained by the photographing device 10. The details of each function will be described below. The processing device 20 may be incorporated in (mounted on) or integrated with the photographing device 10, or may be provided separately from the photographing device 10. If the processing device 20 is provided separately from the photographing device 10, the photographing device 10 and the processing device 20 may be connected to each other wirelessly or by wire, and the photographing device 10 may be remotely operated.

The slit lamp microscope system 1 may also be connected to an outputting device 30. The outputting device 30 displays images obtained by the photographing device 10, and a result of analysis and a result of determination obtained by the processing device 20. The outputting device 30 may be connected to the slit lamp microscope system 1 by wire or wirelessly.

The present embodiment to be described is assumed to have a configuration in which the slit lamp microscope system 1 that integrates the photographing device 10 and the processing device 20 with each other is connected to the outputting device 30.

### 1-2. Photographing Device

### Hardware Configuration

Description will now be made of a hardware configuration of the photographing device 10 with reference to FIG. 1. The photographing device 10 includes an illumination system that scans a human eye with slit light, and a photographing system that captures multiple images at each predetermined angle during the scanning. The photographing device 10 is provided with a hole arranged in front of the eye of a subject. By aligning the hole with the eye so as to cover the eye, the eye can be irradiated with slit light and photographed. By aligning the hole with the eye, the distance between the eye and the photographing device 10 is kept constant. Alternatively, the photographing device 10 may photograph an eye other than a human, or may photograph an eye of various animals such as a dog or a cat.

The illumination system includes a light source 10a, a pinhole 10b, a convex lens 10c, a guide mirror 10d, a slit mechanism 10e, and a scan mirror 10h. An example of the light source 10a is a white light emitting diode (LED). Although visible light is used as the light, infrared light (near-infrared light) may alternatively be used. The light has an intensity that does not cause discomfort to a subject when opening his or her eye. As one example, the light intensity is 1000 nW to 500 nW or less. The present disclosure can obtain a data set that AI can analyze by using system of the present disclosure even if the light is sufficiently weak so as not to cause discomfort to a subject. The quantity of the light emitted from the light source 10a is adjusted by the pinhole 10b, and is focused by the convex lens 10c. The light focused by the convex lens 10c is reflected by the guide mirror 10d and then enters the slit mechanism 10e.

The slit mechanism 10e is formed of, for example, a mechanical slit 10f and a cylindrical lens 10g, and forms slit light. Light reflected by the guide mirror 10d passes through the slit 10f and the cylindrical lens 10g, forming a beam of light (slit light) having a desired size. An example of the slit light having a desired size is an elongated rectangular beam, preferably having a width of 100 to 500 μm and a length of 2 to 3 mm.

With respect to the slit light, the structure of the slit mechanism 10e is not limited to a combination of the mechanical slit 10f and the cylindrical lens 10g, and may employ another mechanism. Alternatively, the slit mechanism 10e may use only the mechanical slit 10f, and form, with a magnifying lens, slit light from light that has passed through the slit 10f. Further alternatively, the slit light may be formed using only the cylindrical lens 10g without the mechanical slit 10f.

The slit light that exits from the slit mechanism 10e is reflected by the scan mirror 10h and is applied to the anterior segment of the eye. The scan mirror 10h includes a motor 10j and its angle is changed in response to the rotation of the motor 10j. Controlling the rotation of the motor 10j changes the angle and the position of incidence of the slit light that impinges on the anterior segment of the eye (hereinafter the anterior segment of the eye may be simply referred to as the eye). The angle of the scan mirror 10h is controlled such that the slit light enters the eye at an angle of 30° to 45°.

The photographing system includes a photographing means (camera) 10i. The photographing means 10i includes a motor 10k, a lens and a photographing element not shown in the drawing. The angle of the photographing means 10i is changed in response to the rotation of the motor 10k. Controlling the rotation of the motor 10k changes the photographing position.

Diagonal irradiation of the anterior segment of the eye with the slit light causes light traveling inside the eye to form a bright line on the surface of the anterior segment of the eye. For example, when the slit light is applied to the eye from the diagonally front-left direction, light impinges on the cornea, the iris, the crystalline lens, and the vitreous body from the left, and a light beam appears at each point. The slit light reflected from the eye passes through the lens of the photographing means 10i and is received by the photographing element. In this way, the eye to be examined is photographed.

The photographing device 10 transmits the obtained images to the processing device 20 during or after the checking, either via a wired or wireless connection. Alternatively, the photographing device 10 may store the obtained images in an internal memory.

For example, the motors 10j,10k are achieved by servo-motors. More preferably, the motors 10j,10k are achieved by stepper motors, which can more precisely control the rotation angles than a servo motor. The rotation angles of the motor 10j,10k are selected to be suitable for scanning and image-capturing the entire anterior segment of an eye.

### Functional Configuration

Next, a description will be given of a functional configuration of the photographing device (photographing unit) 10. The scan mirror 10h of the photographing unit 10 scans the eye with slit light. The initial angle of the scan mirror 10h is set to 20° with respect to the front-rear direction, for example. The initial angle is set in advance such that the slit light impinges on the eye in accordance with the shape of the photographing unit 10. With this initial setting, the slit light can impinge on one end of the eye. Since the average corneal diameter of the eye is about 12 mm, the scanning distance of at least 12mm or more is required to scan the entire eye, including non-corneal areas. For the above, the total scanning distance is preferably set to 15 mm to 25 mm. As an example, the total scanning distance is set to 20 mm. If the total scanning distance is set to 20 mm, setting the angle of the motor 10j that moves scan mirror 10h from the initial angle of 20° to the end angle of 32° makes it possible to move the slit light from the position of one end of the initial setting to the position of the other end of the eye, thereby enabling reliable scanning of the entire cornea. The scan mirror 10h irradiates the eye with the slit light perpendicularly or horizontally according to information required for determining the condition of the eye. The present embodiment assumes that the slit light is applied perpendicularly to the eye. Alternatively, the slit light may be applied horizontally to the eye by rotating the photographing unit 10 by 90° to the left or right.

The photographing unit 10 continuously or discretely changes the angle of the scan mirror 10h, and moving the slit light from one end of an eye to the other end (e.g., from the left end to the right end, or from the upper end to the lower end), and further moves the slit light from the other end back to the one end. This means that the scanning is performed by reciprocating the slit light between the one end and the other end of one eye. A single reciprocating scan takes about 30 seconds. Alternatively, the scanning may be performed in a single direction between the one end and the other end of one eye.

When the scan mirror 10h scans an eye with the slit light, the photographing means 10i moves in synchronization with the scan mirror 10h. In addition, the scan mirror 10h and the photographing means 10i stop at each of predetermined angles during scanning of the eye. This is achieved by controlling the motors 10j and 10k described above. The predetermined angles can be selected from any desired range (e.g., 0.2° to 3°) in the perpendicular and horizontal directions relative to the eye. For example, if the predetermined angle is set to 1°, the eye can be scanned at intervals of 1.67 mm.

In the scanning by the scan mirror 10h, the photographing means 10i captures multiple images at each predetermined angle where the photographing means 10i and the scan mirror 10h stop. The multiple images may comprise any number of frames.

All components of the photographing device 10 may be controlled by the processing device 20 to be described below, or may be controlled by a control device (e.g., an IC board, not shown) provided in the photographing device 10.

### 1-3. Processing Device

### Functional Configuration

A description will now be made of a functional configuration of the processing device (processing unit) 20 illustrated in FIG. 1. The processing unit 20 processes images obtained by the photographing device 10. The processing unit 20 includes at least an analyzing unit 21 that analyzes images obtained by the photographing unit 10 using Artificial Intelligence (AI) and a determining unit 22 that determines the condition of the eye based on results of the analysis by the analyzing unit 21. The processing unit 20 further includes an outputting unit 23 that outputs the obtained images, the result of the analysis, and the results of the determination to the outputting device 30, and a storing unit 24 that stores various data. These components may be implemented by an electronic circuit (hardware), or some of these functions may be provided in hardware, and the remaining may be implemented in the form of software.

The analyzing unit 21 generates an image by averaging the images obtained by the photographing unit 10 and analyzes the generated image using AI. The process of averaging the images serves as a pre-processing step for using the image of the anterior segment of the eye, irradiated with slit light, in AI analysis.

The intensity of the slit light reflected from the eye (in particular, the cornea and the crystalline lens) or diffused inside the eye tends to be weak due to the transparent nature of the cornea and the crystalline lens. Increasing the intensity of the slit light requires recording images and videos in a darker environment, which can cause significant discomfort to the patient. Reducing the intensity of the slit light alleviates the discomfort of the subject, but makes it difficult to detect the slit light reflected from the cornea and the crystalline lens. In addition, images and videos captured at a low intensity of the slit light in dark environment tend to have much noise, which makes it very difficult to obtain images for AI analysis. Thus, image quality and subject comfort are in a trade-off relationship.

With the foregoing in view, the inventors have developed a method that satisfies both image quality and subject comfort. Even when the low-intensity slit light is used, the present method captures multiple images of the reflected slit light at each predetermined angle and selects one or more images, and the photographing means 10i detects the slit light in synchronization with the scan mirror 10h. Accordingly, images with sufficient quality to clearly recognize the cornea and crystalline lens can be obtained. Hereinafter, a description will be given regarding the generation of an image for AI analysis.

### 1-3-1. Generation of an image for AI analysis

The analyzing unit 21 obtains multiple images that the photographing unit 10 has captured at each predetermined angle. The analyzing unit 21 discards one or more images not including a desired eye image from among multiple images (e.g., about 20 images) captured at each predetermined angle. A desired eye image is an image in which the eye is clearly captured. Among multiple images captured at each predetermined angle, the first few images (for example, the first to third frames) are captured immediately after the operation (rotation of the motors) of the scan mirror 10h and the photographing means 10i stop, and are frequently blurred. For the above, the analyzing unit 21 discards the first few images from among the multiple images obtained from the photographing unit 10. In addition, the last few images captured at each predetermined angle, if containing one captured after the operation of the photographing means 10i (rotation of the motors) starts, may be discarded. The analyzing unit 21 may select images by superimposing a rendered image to be described below onto the multiple images obtained from the photographing unit and analyze one or more images that meet a predetermined threshold among the multiple images.

Next, the analyzing unit 21 generates an image (average value image, average image) obtained by taking an average value of the images that have not been discarded among the multiple captured images. By taking the average value of multiple images that have not been discarded, it is possible to obtain an image in which the cornea and the crystalline lens can be clearly recognized even when the intensity of the slit light is low.

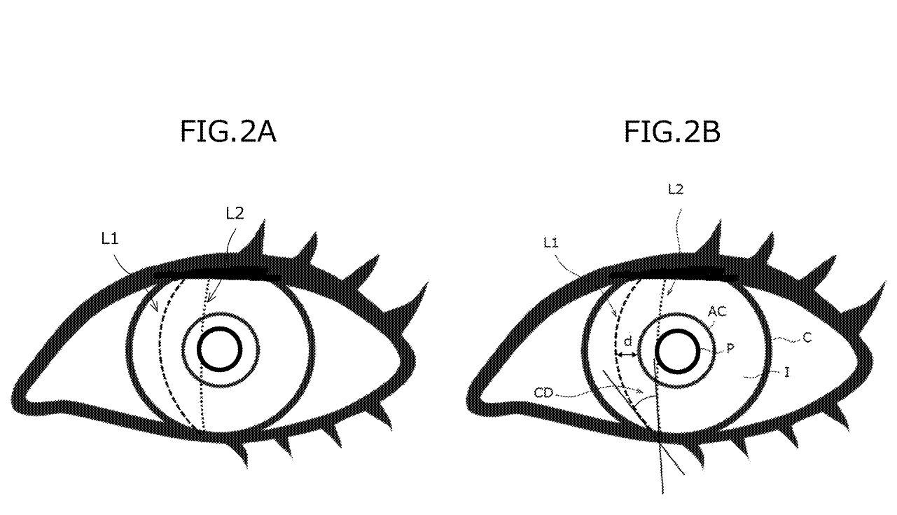

FIG. 2A is a diagram schematically illustrating an image obtained by capturing slit light applied to an eyeball. In FIG. 2A, as an example, the right eye is irradiated with the slit light from diagonally front right. As shown in 2A, the average value image shows the anterior segment of an eye, the slit light L1 passing through (reflected on) the cornea C, and the slit light L2 passing through (reflected on) the iris I. Hereinafter, the slit light L1 is referred to as a corneal reflection slit light L1, and the slit light L2 is referred to as an iris reflection slit light L2.

The analyzing unit 21 obtains one average value image from multiple images captured at each predetermined angle. The analyzing unit 21 performs a process of scanning the eye and simultaneously photographing the eye from one end to the other end of one eye at each predetermined angle, and accordingly generates multiple average value images (the number of times of stopping at respective predetermined angles) in a single scan (at predetermined ranges, for example, one way from the one end to the other end or one lap between the both ends) for one eye.

The analyzing unit 21 stores the multiple average value images as a single video into a memory to be described below. The angle range for which the multiple average value images are generated in the scanning needs to cover the overall area of the eye to be scanned. The angle range can be varied according to the arrangement and the equipment. For example, the overall area of an eye can be photographed by setting the both total scanning angles of the motor 10j and the motor 10k to 12 degrees. In this setting, the eye is photographed by driving the motor 10j and the motor 10k at step intervals of 0.2 to 3 degrees. The step intervals can be changed. The stepwise operation of the motors allows the scan mirror 10h and the photographing means 10i to move by, for example, approximately about 1° per step (which accompanies movement of the slit light by 1.67 mm on the eye per step). The motors stop for approximately 0.5 to 1 second at each step. In each step, the photographing means 10i photographs ten frames. While the slit light makes one lap between one end and the other end, the eye is photographed 50 times (steps). A preferable range of the number of times of photographing per lap may be about 40 to 60 times. The number of frames captured by the photographing means 10i per step may be ten frames or more.

The analyzing unit 21 discards the first to third frames from among the ten frames captured at a predetermined angle in each step, and averages the remaining the fourth to tenth frames to obtain an average value image. The analyzing unit 21 obtains a video including 50 average value images in a single scan, and stores the obtained video in a memory to be described below.

The analyzing unit 21 may further select, from among the generated video, an image in which the slit light impinges on a portion required for determining the condition of the eye. This is because an image in which the slit light is not projected on a portion required for the determination of the condition of the eye is found in an image captured immediately after the start of photographing and also an image captured in the vicinity of the other end of the eye where the scanning light returns. In such images, the slit light is often outside the eye area or the black part of the eye (iris), and is not useful for extracting a feature of the eye.

Since the portion required for determining the condition of the eye is different with a disease, an image of a portion required for determining the condition of the eye may be selected for each disease.

The analyzing unit 21 creates a single video (data set) for AI analysis in the above-described procedures. The analyzing unit 21 stores the data set in the memory described below.

The above processes of selecting (discarding), averaging, and selecting may be accomplished by a general-purpose computer program.

### 1-3-2. Analyzing Process by AI

The analyzing unit 21 causes AI to analyze the average value images included in the data set for AI analysis.

Here, the eye structure is briefly described with reference to FIG. 2B. The particularly critical portions for determining the condition of the eye are the cornea C, the iris I and the crystalline lens AC. For example, the color of the crystalline lens AC is an index of the cataract, the shape/thickness/color of the cornea C become indices of the corneal disorder, and a distance (anterior chamber depth) d from the cornea C to the crystalline lens AC and an angle (iridocorneal angle) CD between the iris I and the cornea C is an index of the glaucoma. In other words, various anomalies of an eye can be detected by identifying the shapes, colors, sizes, and positions of the cornea C, the iris I, and the crystalline lens AC.

In general, an ophthalmologist observes the average value image as shown in FIG. 2A and analyzes the features of the above portions. Qualitative analyses, which rely on the experience of an ophthalmologist, are relatively fast, but at this stage, can only screen for normality or abnormalities.

If the analysis reveals that subject is suspected of having a disorder, the subject undergoes a detailed check using AS-OCT (Anterior Segment Optical Coherence Tomography) to obtain quantitative information on the feature of the portion. The device AS-OCT is relatively expensive and is not readily available in small hospitals or clinics.

The type and the grade of cataract can be determined by observing the color of the crystalline lens AC appearing on the average value images even if the shape of the crystalline lens AC is unclear to some extent. For example, it can be determined that a transparent crystalline lens AC represents normal, a yellow crystalline lens AC represents mild cataract, and a yellow-green crystalline lens AC represents severe cataract.

On the other hand, the corneal thickness C, the iridocorneal angle CD, and the anterior chamber depth d are quantitative information obtained from the corneal reflection slit light L1 and the iridocorneal reflection slit light L2 appearing in the average value images, and are difficult to obtain from only observations.

In order to obtain quantitative information on the corneal thickness C, the iridocorneal angle CD and the anterior chamber depth d at a low cost, a general-purpose image processing program, such as an Image J, can be used. The corneal thickness C, the iridocorneal angle CD, and the anterior chamber depth d can be obtained manually by using this program, but several steps are required to process a single image, which takes much time and requires labor. In particular, it is difficult to generate a rendered image of the corneal thickness C.

With the foregoing problems in view, the present inventors propose a method of autonomously extracting the features (e.g., corneal thickness C, iridocorneal angle CD, and anterior chamber depth d) required to determine the condition of an eye from an image by applying the lightweight AI algorithm developed by the inventors.

The analyzing unit 21 converts the obtained image into a rendered image representing feature portions of the corneal, the iris and the pupil. Specifically, the analyzing unit 21 converts the average value image into a rendered image representing feature portions of the cornea C, the iris I, and the pupil P, and outputs the rendered image.

The lightweight AI algorithm is configured to segment a target portion from a biometric image obtained by photographing and denoising (i.e., forming into a high-resolution image) the captured image. The lightweight AI algorithm is based on a Unet, which is a deep learning (DL) model designed for biomedical image segmentation, and is augmented with attention blocks in the contraction, expansion, and bottleneck paths, and introduces continuous narrowing of the channels toward the middle block (bottleneck). This network is referred to as a lightweight bottleneck narrowing Unet (LWBNA_UNet) with attention blocks. A detailed description of the lightweight AI algorithm is incorporated herein by reference in International Publication Pamphlet No. WO 2023/062764.

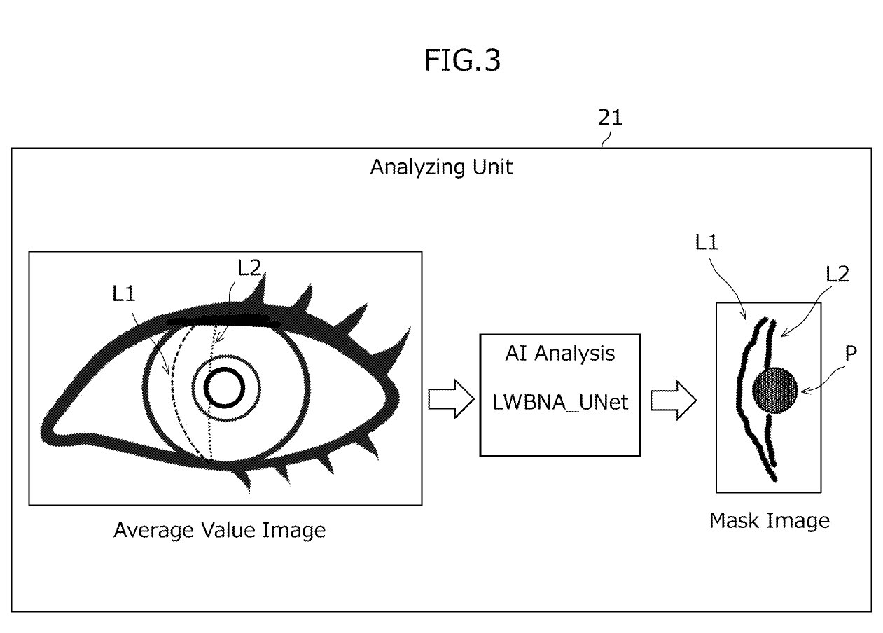

FIG. 3 is a diagram illustrating a process in which the analyzing unit extracts features from an average value image using LWBNA_UNet. The analyzing unit 21 inputs the generated data set into LWBNA_UNet. FIG. 3 illustrates an example in which a frame (hereinafter, referred to as a generated frame) of the generated average value images in the data set is input.

The LWBNA_UNet adds an attention block that averages channels to a convolution block, so that the image size is reduced and feature points are extracted. Further, the LWBNA_UNet may extract the feature points by preferentially reducing a region that does not include the outer shape of the segmentation target site included in the average value image.

The LWBNA_UNet extracts information related to a feature image obtained by segmentation of the average value image including the extracted features by contracting the channel under a state where an attention block is added to the convolution block. After contracting the channel, the analyzing unit 21 may expand the feature image to a size the same as the input average value image and then output the expanded feature image.

In the present embodiment, the LWBNA_UNet finds the center of the pupil P shown in FIG. 2 from a generated frame of an average- value image, sets the pupil P at the center of the generated frame, detects features point in the region of interest (the pupil P, the corneal reflection slit light L1, and the iris reflection slit light L2), and outputs a rendered image representing features of the region of interest (right drawing of FIG. 3). The region of interest in the rendered image may be color-coded by area. The rendered image may be superimposed on the average value image and may be referred to as a mask image. In this process, after aligning one or more frames in which the position of the pupil P coincides with the center of the mask image, respective generated frames are sorted on the basis of a distance from the center of the pupil from an edge of the cornea.

An image of each generated frame obtained after processing by the AI may be selected, trimmed, and aligned, for example. The aligned image follows the position of the scanning slit light in the eye. As such, these images can be used in conjunction with the mask image to analyze a variety of eye diseases. For example, images of glaucoma can be used. In addition, the angle between the iris and the cornea can also be calculated at different position of the eye, the thickness of the cornea can be measured from each frame image at different angle positions, and a map representing the cornea thickness can also be generated by merging the measurements of all generated frame images.

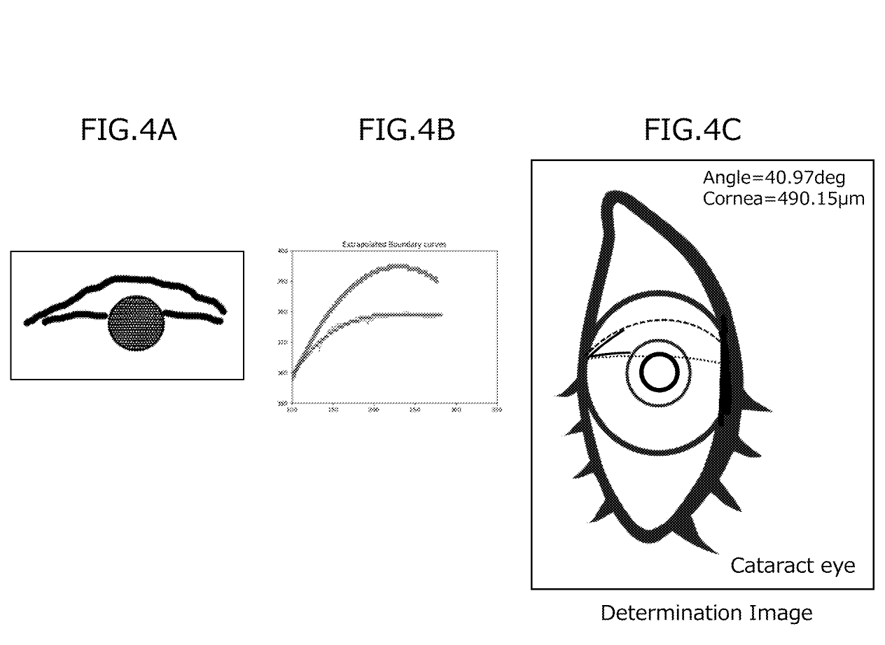

Assuming that the average value image is an image obtained by projecting the slit light perpendicular to the eye, the average value image is rotated clockwise by 90° about the pupil and then input into the LWBNA_UNet. The purpose of this process is to facilitate a process in the program (e.g., python) used to generate the graph (see FIG. 4B) to be described below. Here, the average value image input into LWBNA_UNet and the rendered image (mask image) to be output are ones obtained by rotating the average value image shown in FIG. 3 by rotating clockwise by 90° about the pupil.

### 1-3-3. Obtaining Process of 3D Information

The analyzing unit 21 obtains three-dimensional information of the eye from the rendered image. In other words, the analyzing unit 21 calculates various parameters of the regions of interest from the mask image. Examples of the various parameters are the iridocorneal angle (anterior chamber angle) and the corneal thickness, and the present embodiment describes the iridocorneal angle CD and the corneal thickness C as the parameters.

FIG. 4 is a diagram illustrating a process in which the analyzing unit 21 calculates various parameters. FIG. 4A illustrates an average value image obtained by rotating the mask image shown in FIG. 3 counterclockwise by 90° about the pupil and expressed in a two-dimensional array of the numerical values (pixels) in the X-Y direction. FIG. 4B is a graph that plots the boundary between the corneal and the iris obtained from FIGS. 4A, and 4C is an image in which the condition of the eye is determined.

Calculation of the iridocorneal angle CD and the corneal thickness C from FIG. 4A requires coordinates (x,y) of an outer edge (distal from the pupil) of the cornea, the inner edge (proximal from the pupil) of the cornea, the outer edge (distal from the pupil) of the iris. The coordinates are obtained by applying an X–Y grid to FIG. 4A, determining coordinates of lower-limit positions of corneal reflection slit light L1 and iris reflection slit light L2 in the mask image, the positions being indicated by pixel-based curves in FIG. 4A, and fitting the coordinates with nonlinear curves. As a result, a graph in which the respective slit lights are plotted as dots is generated (FIG. 4B). The horizontal axis and the vertical axis of FIG. 4B respectively indicate pixels in the X direction and the Y direction of the captured image. The fitting may use existing software. As shown in FIG. 4B, generating the graph by fitting the nonlinear curves makes it possible to estimate the positions of pixels (e.g., junction between the cornea and the iris) that are not present in the mask image. This allows determination of an angle between the two curves, the angle between the two curves corresponding to the iridocorneal angle CD, and a thickness of an upper curve corresponding to the corneal thickness C.

The analyzing unit 21 calculates (measures) the iridocorneal angle CD and the corneal thickness C from the coordinates of the graph in FIG. 4B. The analyzing unit 21 stores the generated graph and the calculated values into the memory described below. The three-dimensional (3D) information of the eye obtained by the analyzing unit 21 is referred to as an analysis result.

### 1-3-4. Determination Process of Eye Condition

The determining unit 22 determines the condition of an eye based on the analysis result from the analyzing unit 21. Specifically, the determining unit 22 determines (evaluates and estimates) the condition of the eye by comparing the calculated value with a predetermined threshold for each symptom (disorder) of an eye. Examples of the condition (disorder) of an eye include cataracts, glaucoma, keratoconus, and amyloidosis.

The determining unit 22 weights an image captured at an optimum predetermined angle for determining a condition of an eye, and then determines the condition of the eye. The determining unit 22 selects an average value image exhibiting a particular symptom (disorder) of the eye, and adds the subject's condition (attribute and medical history) as a weight to determine the condition of the eye.

The determining unit 22 generates a determination image. As an example, the determining unit 22 may generate a determination image by superimposing the calculated parameters and the determination result onto an average-value image at each predetermined angle. The determining unit 22 may further generate the determination image exhibiting two supplemental lines indicating the iridocorneal angle CD. FIG. 4C is an image (determination image) in which the condition of the eye is determined. FIG. 4C indicates an iridocorneal angle CD of “Angle = 40.97 deg” and a corneal thickness C of “Cornea = 490.15 μm” in an upper right portion, exhibits “Cataract eye” as a result derived from these values, and also exhibits two auxiliary lines representing the iridocorneal angle CD.

The determining unit 22 may calculate, as the three-dimensional information, a color value of the crystalline lens from the average value images and determine the condition of the eye by comparing the calculated value with a predetermined threshold. This is particularly useful in determining the symptom of cataracts.

The determining unit 22 stores, for example, the generated graph, the calculated values, the determination result, and the determination image in a memory to be described below.

The outputting unit 23 outputs the determination result by the determining unit 22 to the outputting device 30. The outputting device 30 may be provided separately from the processing device 20, or may be incorporated (mounted) in or integrated with the processing device 20. Examples of the outputting device 30 include a monitor, a projector, and a printer. For example, a monitor may be provided and configured to output an image captured by the photographing device 10, or to output an average value image, a rendered image, a generated graph, three-dimensional information generated or calculated by the analyzing unit 21 of the processing device 20, and a determination result from the determining unit 22. The determination result may be visually presented on a screen of the monitor connected to the processing device 20 or may be audibly notified by voice, for example.

The storing unit 24 stores an image obtained from the photographing device 10, an average- value image, a rendered image, and a generated graph and the three-dimensional information generated or calculated by the analyzing unit 21, and a determined results by the determining unit 22, and information of various data (threshold for each condition of the eye) used for the determination. Furthermore, the storing unit 24 may store data indicating a number of examinations and subject information, such as attributes including age, gender, height, weight, and body type.

### Hardware Configuration

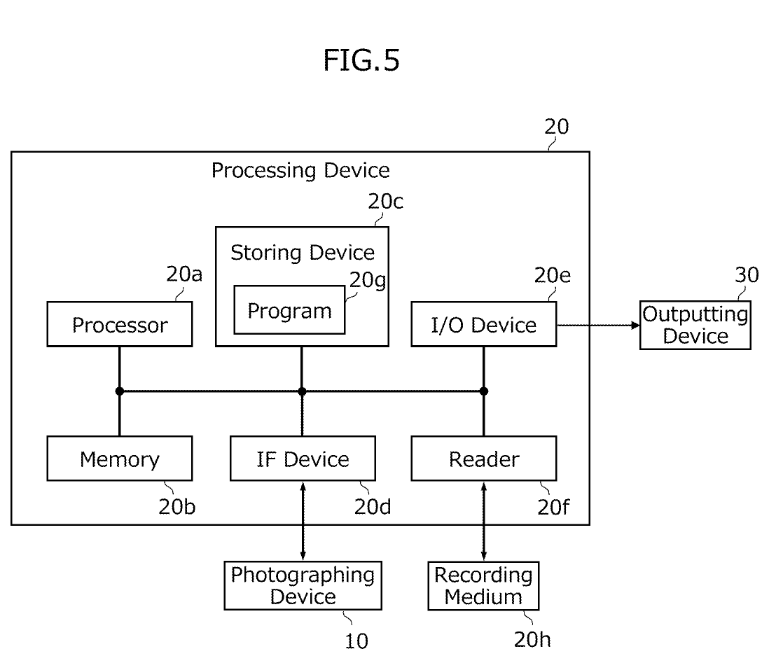

FIG. 5 is a diagram illustrating an example of a hardware (HW) configuration of a computer that implements functions of the processing device 20. Hereinafter, the processing device 20 is also referred to as a “computer 20”. The slit lamp microscope system 1 of the present embodiment is achieved by a general-purpose computer 20 capable of executing a program 20g (analysis and determination program) for analyzing and determining images. As shown in FIG. 5, the computer 20 illustratively includes a processor 20a, a memory 20b, a storing device 20c, an IF device 20d, an IO device 20e and a reader 20f. These components are communicably connected to one another via a bus (e.g., control bus, data path) provided within the computer 20.

The processor 20a is an example of an arithmetic processing device that performs various controls and calculations. The processor 20a may be communicably connected to the respective blocks in the computer 20 via the bus. Examples of the processor 20a include an Integrated Circuit (IC), such as a Central Processing Unit (CPU) and a Graphics Processing Unit (GPU).

The memory 20b and the storing device 20c are examples of hardware (HW) devices that store various types of data and information such as a program. The storing device 20c may store a program 20g that implements all or a part of various functions of the processing device 20. The program 20g may be expanded in the memory 20b. The memory 20b and the storing device 20c serve as an example of the storing unit 24 shown in FIG. 1.

The IF device 20d is an example of a communication IF that controls connection and communication with a network. For example, the IF device 20d may include adapters conforming to Ethernet (registered trademark), InfiniBand, Myrinet, or optical communications such as FCs (Fibre Channel), and the like. The adapters may be compatible with either or both wireless and wired communication schemes. The program 20g may be, for example, downloaded from the network to the computer 20 via the communication IF and stored in the storing device 20c.

The hardware is configured to be capable of transmitting data to a specialist for remote control and/or diagnosis.

The I/O device 20e is circuitry and/or software that inputs external data and external signals into a device or a system and outputs data or signals to an external device. The I/O device 20e serves as an example of the outputting unit 23 shown in FIG. 1. The I/O device 20e may include one or both of an input device and the output device 30. Examples of the input device include a keyboard, a mouse, and a touch panel, and examples of the output device 30 include those described above.

The reader 20f is an example of a reader (e.g., adapter, drive device, card reader) that reads data and programs recorded on a non-transitory recording medium 20h. The reader 20f may include a connecting terminal or device to which the recording medium 20h can be connected or inserted. The program 20g may be stored on the recording medium 20h, and the reader 20f may read the program 20g from the recording medium 20h and store the read program 20g into the storing device 20c.

The processor (processor circuitry) 20a of the present embodiment achieves the functions (analyzing unit (analyzing processor circuitry) 21, determining unit (determining processor circuitry) 22) of the processing device 20 illustrated in FIG. 1 by loading the program 20g (analysis and determination program) stored in the storing device 20c into the memory 20b and executing the expanded program 20g. In addition, the processor 20a may construct a machine learning model using hardware circuitry, a virtual network implemented by the processor 20a via the program, or general-purpose software.

The storing device 20c may further store a program (control program) for controlling the photographing device 10. The computer 20 may be connected to the IF device (not shown) of the photographing device 10 and communicate therewith via the IF device 20d and remotely control the photographing device 10 by expanding the control program on the memory 20b and executing the expanded program. All or part of the program for controlling the photographing device 10 may be included in the above-described analysis and determination program 20g.

The processor 20a obtains data (hereinafter also referred to as “requisite data”) required to implement the functions of the computer 20 based on data received via the IF device 20d, input from the input device of the I/O device 20e, data read by the reader 20f, and data stored in the storing device 20c.

The above required data may be stored in advance in the memory 20b and the storing device 20c, or may be input via the input device of the I/O device 20e. The requisite data includes an image obtained from the photographing device 10, an average value image, a rendered image a graph, and the three-dimensional information generated or calculated by the analyzing unit 21, and a result of determination by the determining unit 22, and information of various data (thresholds for each condition of the eye) used for the determination. In addition, the requisite data may include data of the number of examinations and the information of the subject (attributes such as age, gender, height, weight, and body type).

A signal required to control the photographing device 10 may be input via the input device of I/O device 20e. This signal includes the signal that triggers an operation of the photographing device 10, such as the start of detection and the end of detection.

The HW configuration of the computer 20 (processing device 20) described above is exemplary. Accordingly, the processing device 20 may undergo appropriate modification, including addition or removal of hardware components (e.g., addition or deletion of arbitrary blocks), division, integration in any combination, and addition or deletion of the bus.

The HW configuration of the computer 20 (processing device 20) can be implemented with a high-performance single-board computer referred to as a small edge computing device, such as Rasberry Pi (registered trademark) or Jetson (registered trademark). In the present embodiment, Jetson Nano or Jetson Xavier (registered trademark) is used.

### 2. Method for Operating Slit Lamp Microscope System

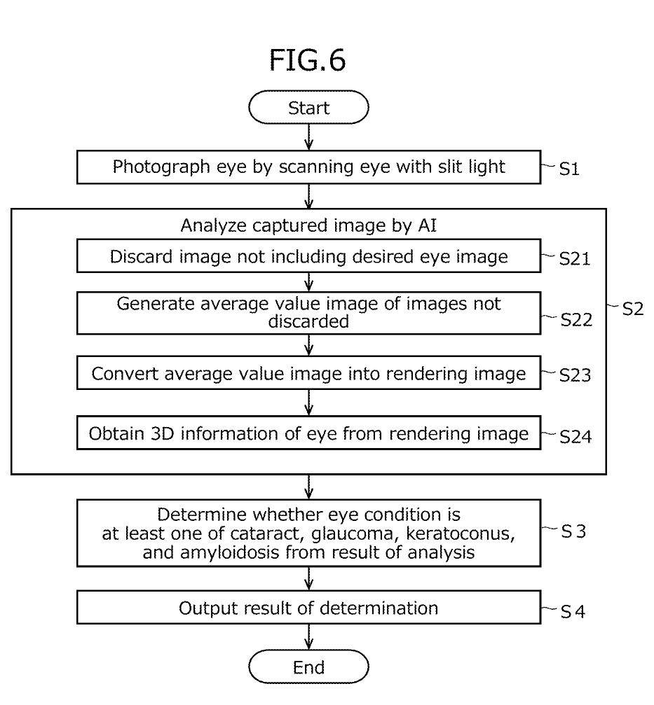

FIG. 6 is an example of a flow chart illustrating a procedure to operate the slit lamp microscope system according to the embodiment, and shows the details of the processes performed by the photographing device 10 and the processing device 20. Step S1 to be described below can be achieved by controlling the photographing device 10 by the control program. Steps S2 and S3 to be described below can be achieved by the processing device 20 when the analysis and determination program 20g causes the processing device 20 to execute these corresponding processes. The following description assumes that this flow chart starts at a timing when the photographing device 10 starts capturing images.

In Step S1, the photographing device 10 captures images of the eye by scanning the eye with slit light (photographing step). The scan mirror 10h of the photographing device 10 scans the eye with the slit light. During scanning by the scan mirror 10h, the photographing means 10i moves in synchronization with the scan mirror 10h. At each predetermined angle in the scanning, the photographing means 10i captures multiple images of the eye. In other words, the photographing means 10i captures multiple images when it stops at a predetermined angle. The photographing means 10 transmits the captured images to the analyzing unit 21.

In Step S2, the analyzing unit 21 analyzes the images obtained from the photographing device 10 with AI (analyzing step). First, the analyzing unit 21 discards one or more images that do not include a desired eye image from among the obtained images (Step S21). Next, the analyzing unit 21 generates an average value image of the images that have not been discarded (Step S22). The analyzing unit 21 obtains one average value image for multiple images captured at each predetermined angle. The analyzing unit 21 obtains multiple average value images (corresponding to the number of stops at the respective predetermined angles) in a single scan for one eye. The analyzing unit 21 generates a data set for AI analysis in the form of a single video containing multiple obtained average value images.

The analyzing unit 21 converts the average value image into a rendered image (Step S23). The analyzing unit 21 extracts features from the average value image using LWBNA_UNet. LWBNA_UNet identifies a center of a predetermined region from an average value image, sets the predetermined region at the center of the generated frame, detects feature points of a region of interest, and outputs a rendered image representing the feature of the region of interest.

The analyzing unit 21 obtains three-dimensional (3D) information of the eye from the rendered image (Step S24). The analyzing unit 21 generates a graph by fitting the predetermined region of the rendered image with a non-linear curved line. The analyzing unit 21 calculates various parameters from coordinates of the generated graph.

In Step S3, the determining unit 22 determines the condition of the eye on the basis of the result of the analysis by the analyzing unit 21 (determination step). The determining unit 22 determines whether the condition of the eye corresponds to at least one of cataract, glaucoma, keratoconus, and amyloidosis on the basis of the result of the analysis by the analyzing unit 21. The determining unit 22 determines the condition of the eye by comparing the 3D information that the analyzing unit 21 calculates with a threshold for each eye condition (disorder) and generates the result of the determination.

In Step S4, the outputting unit 23 outputs the result of determination generated by the determining unit 22 (output step). The outputting unit 23 displays, on the outputting device 30, a determination image obtained by superimposing the three-dimensional information of the eye calculated by the analyzing unit 21 and the result of the determination by the determining unit 22 onto the average value image at each predetermined angle. After the result of the determination is recorded in the storing unit 24, the process of this flow ends.

### 3. Action and Effect

(1) In the above-described slit lamp microscope system 1, the analysis and determination program, the non-transitory computer-readable recording medium having stored therein the analysis and determination program, and the method of operating the slit lamp microscope system, images obtained by scanning the eye with slit light are analyzed by AI, and the condition of the eye is determined on the basis of a result of the analysis. The obtained images include multiple images captured at each of predetermined angles in the scanning. This makes it possible to obtain images containing the 3D position information of the region of interest of the eye and being in a state analyzable by AI. Accordingly, the obtained images of the eye can be analyzed rapidly and accurately through the analysis using AI. In addition, determination using the result of analysis made by the AI can determine the condition of the eye easily at a low cost. This enables simple checking and early detection of a possible abnormality without going to a hospital, and allows a disorder of the eye to be treated before it becomes severe through a detailed examination at a hospital, thereby contributing to an improved expectation of patient recovery.

(2) In the above-described technique, the photographing device (photographing unit) 10 captures images. The photographing device 10 includes the light source 10a, the cylindrical lens 10g that forms slit light, the scan mirror 10h that scans an eye, and photographing means 10i. In scanning, the scan mirror 10h and the photographing means 10i move in synchronization with each other. The photographing device (photographing unit) 10 is inexpensive due to its simple configuration, and therefore can be introduced in a wide range of facilities. In addition, since the scan mirror 10h and the photographing means 10i move in synchronization with each other, the eye can be accurately photographed.

(3) In the above-described technique, the scan mirror 10h and the photographing means 10i synchronously stop at each predetermined angle, and the photographing means 10i captures multiple images while stopped. This makes it possible to obtain accurate images capturing the entire surface of the eye.

(4) In the above-described technique, the analyzing unit 21 performs analysis. The analyzing unit 21 discards one or more images not including a desired eye image from among multiple captured images, and generates an image by taking an average value of the images that have not been discarded among the multiple captured images. By discarding the first two or three frames from the captured images, the blurred image captured immediately after the start of photographing can be eliminated. In addition, by taking an average value of the multiple images that have not been discarded, an image in which the cornea and the crystalline lens are clearly visible can be obtained even when the light amount of the slit light is small.

(5) In the above-described technique, the analyzing unit 21 converts the obtained image into a rendered image representing feature portions of the cornea, the iris and the pupil. This can accomplish check by means of AI without applying a large load to the CPU, so that the device can be avoided from increasing its size and can enhance the processability of images required for determining respective eye conditions.

(6) In the above-described technique, the analyzing unit 21 obtains three-dimensional information of the eye from the multiple rendered images obtained at respective predetermined angles. This can obtain information quantitatively indicating the condition of the eye.

(7) In the above-described technique, the determining unit 22 performs determination. The determining unit 22 determines whether the condition of the eye is at least one of cataract, glaucoma, keratoconus, and amyloidosis on the basis of the result of the analysis by the analyzing unit 21. Thus, the condition of the eye can be specified.

(8) In the above-described technique, in determining the condition of the eye, the determining unit 22 weights an image captured at an optimum predetermined angle for the condition of the eye to be determined and then determines the condition of the eye. This makes it possible to determine the condition of the eye more accurately.

In the above-described technique, the conversion to a rendered image uses a lightweight AI algorithm (LWBNA_UNet) that adds an attention block to an algorithm (UNet) for image segmentation and introduces continuous narrowing of a channel to a middle block. This makes it possible to obtain a rendered image easily with fewer procedures and time than manual generation of a rendered image.

### 4. Miscellaneous

The configurations of the photographing device 10 and the processing device 20 described above are exemplary, and are not limited thereto. Description of the above-described embodiment presumes the photographing device 10 transmits the images to the processing device 20 in real time during photographing. In this case, the analysis and the determination in the processing device 20 may also be performed in real time. Alternatively, the present disclosure is not limited thereto, and the photographing device 10 may store the photographed image in the memory, and the processing device 20 may obtain the photographed image after the photographing is finished. In this alternative, the analysis and the determination in the processing device 20 may also be performed after the images are obtained after the end of the photographing.

The determining unit 22 of the processing device 20 may record the result of the determination of a subject and compare the result of the current determination with the results of the past determinations so that the degree of progress of the condition of the eye can be determined. In this alternative, the determination image may exhibit the degree of progression of the eye condition.

As one aspect, it is possible to obtain eye images suitable for AI analysis by photographing with an inexpensive and simple device, and to rapidly and accurately analyze the images using AI to determine the condition of the eye.

Throughout the descriptions, the indefinite article “a” or “an”, or adjective “one” does not exclude a plurality.

All examples and conditional language recited herein are intended for the pedagogical purposes of aiding the reader in understanding the disclosure and the concepts contributed by the inventor to further the art, and are not to be construed limitations to such specifically recited examples and conditions, nor does the organization of such examples in the specification relate to a showing of the superiority and inferiority of the disclosure. Although one or more embodiments of the present disclosures have been described in detail, it should be understood that the various changes, substitutions, and alterations could be made thereto without departing from the spirit and scope of the disclosure.

What is claimed is:

1. A slit lamp microscope system comprising:
a photographing device that scans an eye with slit light;
a memory; and
processor circuitry coupled to the memory and configured to
analyze an image obtained by the photographing device using Artificial Intelligence (AI), and
determine a condition of the eye based on a result of the analysis; wherein
the photographing device captures a plurality of images at each of predetermined angles during the scanning at least one way from one end to other end of the eye.

2. The slit lamp microscope system according to claim 1, wherein
the photographing device comprises a light source, a lens that forms the slit light, a scan mirror that scans the eye, and a camera, and
the camera photographs the eye while moving in synchronization with the scan mirror.

3. The slit lamp microscope system according to claim 2, wherein
the scan mirror and the camera stop at each of the predetermined angles and the camera photographs the plurality of images when the scan mirror and the camera stop at each of the predetermined angles.

4. The slit lamp microscope system according to claim 1, wherein
the processor circuitry discards one or more images not including a desired eye image from among the plurality of images, and generates an image averaging one or more images of the plurality of images not discarded.

5. The slit lamp microscope system according to claim 1,
the processor circuitry converts the obtained image into a rendered image representing feature portion of a cornea, an iris, and a pupil.

6. The slit lamp microscope system according to claim 5, wherein
the processor circuitry obtains three-dimensional information of the eye from a plurality of the rendered images obtained one at each of the predetermined angles.

7. The slit lamp microscope system according to claim 1, wherein
the processor circuitry determines whether the condition of the eye is at least one of cataract, glaucoma, keratoconus, and amyloidosis from a result of the analyzing.

8. The slit lamp microscope system according to claim 1, wherein
the processor circuitry weights an image at an optimum angle for the condition of the eye to be determined among the predetermined angles in the determining the condition of the eye.

9. A non-transitory computer-readable recording medium having stored therein computer readable instructions, which, when executed by processor circuitry, cause the processor circuitry to perform a method, the method comprising:
analyzing an image obtained by a photographing device using Artificial Intelligence (AI), and
determining condition of an eye based on a result of the analysis, wherein
the photographing device scans the eye with slit light and captures a plurality of the images at each of predetermined angles during the scanning at least one way from one end to other end of the eye.

10. A computer-implemented method for operating a slit lamp microscope system comprising a photographing device, a memory, and a processor circuitry coupled to the memory, the method comprising:
scanning an eye with slit light at the photographing device,
at the processor circuitry,
analyzing an image obtained by the photographing device using Artificial Intelligence (AI), and
determining a condition of an eye based on a result of the analysis, wherein
the photographing device captures a plurality of the images at each of predetermined angles during the scanning at least one way from one end to other end of the eye.
